(19) United States
(12) Patent Application Publication
(10) Pub. No.: US 2024/0173532 A1
(43) Pub. Date: May 30, 2024

(54) Male Disinfection Cap

(71) Applicant: Becton, Dickinson and Company, Franklin Lakes, NJ (US)

(72) Inventors: Shishir Prasad, Ramsey, NJ (US); Praveen Nalawade, Belagavi (IN)

(21) Appl. No.: 17/994,974

(22) Filed: Nov. 28, 2022

(51) Int. Cl. A61M 39/16; A61M 39/20

(52) U.S. Cl. CPC A61M 39/162; A61M 39/165; A61M 39/20; A61M 2207/10

(57) ABSTRACT

A cap configured to engage a male connector includes a housing having an open first end, a second end, and at least one sidewall extending between the first end and the second end. The housing also includes a protrusion configured to engage the male connector for retaining the male connector within the housing and a center post for sealing a lumen in the male connector. The cap also includes an outer absorbent support disposed in the recess between the sidewall of the housing and the protrusion and an inner absorbent support disposed in the recess between the protrusion and the center post configured to contain a cleaning solution for cleaning and/or disinfecting portions of the male connector including the tip of the male connector engaged to the cap.

### BACKGROUND OF THE INVENTION

### Field of the Invention

The present disclosure relates generally to caps for medical connectors and, in particular, to a medical cap configured to be attached to male connectors for sealing, cleaning, and disinfecting critical portions of the male connectors, including luer connections.

### Description of Related Art

Vascular access devices (VADs) are commonly used medical devices, which can include intravenous (IV) catheters, such as peripheral catheters or central venous catheters. If not properly maintained or if exposed to a non-sterile environment, the VADs can become contaminated, sealed with blood clots, and/or can spread infection. Further, bacteria and other microorganisms may enter into a patient's vascular system from access hubs, ports, or valves upon connection to the VAD to deliver a fluid or pharmaceutical to a patient. Therefore, each access hub, port, valve, or other connection configured for attachment to a VAD is associated with some risk of transmitting a catheter related bloodstream infection (CRBSI) to a patient.

Many medical facilities implement sterile practices and protocols to ensure that VADs and access hubs or ports are used properly and do not become soiled or infected. These protocols often include sterilizing the access hubs, ports, and VADs, as well as flushing the catheter with a flush solution prior to use. Specifically, VAD standards of practice usually recommend flush procedures be performed after catheter placement, before fluid infusion, and before and after drug administration, blood sampling, transfusions, and/or administration of parenteral nutrition.

Standards of practice can also require that access hubs, ports, and valves be capped with disinfection caps when not in use, to prevent microbial ingress into the hub, port, or valve and to sterilize areas of the hub, port, or valve that contact the VAD. Disinfection caps are disposable cap devices that contain an amount of cleaning or disinfecting solution for sterilizing portions of the port, hub, and valve. Accordingly, disinfecting caps can be both a barrier preventing microbes, particles, and debris from contacting portions of hubs, ports, or VADs that should remain sterile and disinfecting or cleaning devices configured to expose portions of the hubs, ports, and VADs to cleaning and disinfecting solutions.

However, conventional disinfecting caps may only clean or disinfect only portions of a male connector. Conventional methods with wipes and caps may not clean and disinfect other critical surfaces of the male connector in order to ensure that critical surfaces of the distal region of the male connector remains sterile. The disinfecting caps of the present disclosure are configured to address these issues by, for example, including an IPA sponge that comes in direct contact with and disinfects the tip of a male luer connector and a center post to prevent IPA ingress into the male luer connector.

### SUMMARY OF THE INVENTION

According to an aspect of the disclosure, a cap configured to engage a male connector includes a housing having an open first end, a second end, and a sidewall extending between the first end and the second end and having an inner and an outer surface defining a first cylindrical recess or cavity. The housing also includes a protrusion extending from the second end of the housing into the first recess and also having a front open end and a sidewall, which can be essentially cylindrical, extending between the front open end of the protrusion and the second end of the housing and having an inner and an outer surface defining a second cylindrical recess or cavity that is of a smaller diameter than the first recess defined by the housing. The protrusion is configured to engage the male connector for retaining the male connector within the housing, for example by the use of threads or luer lugs extending outward from the outer surface of the sidewall of the protrusion adjacent the front open end of the protrusion. The housing further includes a center post extending from the center of the second end of the housing into the second recess defined by the protrusion. The center post is configured to prevent ingress of disinfectant into the lumen of the male connector during disinfection process. The cap also includes an outer absorbent support that is cylindrical in shape and that defines a central cylindrical opening that is configured to be inserted into the first recess of the housing between the inner surface of the housing sidewall and the outer surface of the protrusion sidewall. The outer absorbent support is configured to clean and/or disinfect portions of the male connector such as the shield of a male luer connector. The cap also includes a cylindrical inner absorbent support that is cylindrical in shape and that defines a central cylindrical opening that is configured to be inserted into the second recess of the protrusion between the inner surface of the protrusion sidewall and the outer surface of the center post. The inner absorbent support is configured to contain cleaning solution for cleaning and/or disinfecting portions of the male connector engaged to the cap, including the distal end or tip of a stem of the male connector.

According to another aspect of the disclosure, a method for attaching the previously described cap to the male connector includes a step of inserting a distal end of a stem of the male connector through the open first end of the housing into the first recess formed by the housing and the second recess formed by the protrusion causing the distal end of the stem to contact a scal disposed in the protrusion and the inner absorbent support and causing the distal end of the annular shield of the male connector to contact the outer absorbent support. The method also includes a step of rotating the male connector relative to the housing causing the stem of the male connector to advance through the second recess axially compressing the inner absorbent support and the annular shield of the male connector to axially compress the outer absorbent support.

According to another aspect of the disclosure, a manufacturing method for the previously described cap includes steps of inserting the inner absorbent support into the second recess formed between the inner surface of the sidewall of the protrusion and the center post and inserting the outer absorbent support into the housing into the first recess formed between the outer surface of the sidewall of the protrusion and the inner surface of the sidewall of the housing.

In accordance with an embodiment of the present invention, a cap is configured to engage a male connector, the cap including a housing comprising an open first end, a closed second end, and a sidewall extending between the first end and the second end; a protrusion extending from the closed second end of the housing towards the open first end of the housing configured to engage the male connector for retaining the male connector within the housing and defining a first recess between the sidewall of the housing and the protrusion; a center post extending from the closed second end of the housing towards the open first end of the housing configured to engage the male connector and defining a second recess between the protrusion and the center post; an outer absorbent support disposed in the first recess configured to contain a cleaning solution for cleaning and disinfecting portions of the male connector engaged to the cap; and an inner absorbent support disposed in the second recess configured to contain a cleaning solution for cleaning and disinfecting portions of the male connector engaged to the cap.

In accordance with an embodiment of the present invention, the male connector includes a luer stem including a tip and defining a central lumen and an annular shield extending about the stem, wherein the shield of the male luer connector comprises threads on an inner surface of the shield configured to engage corresponding threads of a female luer connector for securing the male connector to the female luer connector, and wherein the protrusion comprises an outer surface and threads on the outer surface of the protrusion configured to engage the threads on the inner surface of the shield of the male connector thereby releasably securing the male connector to the cap.

In accordance with an embodiment of the present invention, when the male connector is engaged to the cap, the luer stem of the male connector is inserted into the second recess and in contact with the inner absorbent support, the center post is inserted into the central lumen of the male connector, and the annular shield of the male connector is in contact with the outer absorbent support.

In accordance with an embodiment of the present invention, when the male connector is engaged to the cap, cleaning solution is exposed to the threads on the inner surface of the annular shield for cleaning and disinfecting the threads, cleaning solution is exposed to the outer surface of the luer stem for cleaning and disinfecting the outer surface of the luer stem; and the center post blocks cleaning solution from entering the central lumen of the male connector.

In accordance with an embodiment of the present invention, the cap further includes a protective cover configured to be affixed to the open first end of the housing to prevent exposure of the protrusion, center post, outer absorbent support, and inner absorbent support to contamination prior to use of the cap.

In accordance with an embodiment of the present invention, the protrusion further includes a thread extending radially outward from the sidewall of the protrusion, the thread configured to engage threads on an inner surface of a shield of the male connector.

In accordance with an embodiment of the present invention, the engagement between the thread of the protrusion and the threads of the shield of the male connector rotatably secures the male connector within the housing.

In accordance with an embodiment of the present invention, the sidewall of the protrusion includes at least one opening for permitting the cleaning solution to pass from the inner absorbent support through the protrusion to the outer absorbent support.

In accordance with an embodiment of the present invention, insertion of the male connector into the second recess formed between the protrusion and the center post causes the inner absorbent support to axially compress farther into the second recess, and wherein the axial compression of the inner absorbent support expels the cleaning solution from the inner absorbent support causing the cleaning solution to move through the second recess contacting an outer surface of a stem of the male connector inserted in the second recess including the tip of the stem of the male connector.

In accordance with an embodiment of the present invention, wherein insertion of the male connector into the first recess formed between the housing and the protrusion causes the outer absorbent support to axially compress farther into the first recess, and wherein the axial compression of the outer absorbent support expels the cleaning solution from the outer absorbent support causing the cleaning solution to move through the first recess contacting a shield of the male connector inserted in the first recess.

In accordance with an embodiment of the present invention, wherein the central post is configured to seal an open end of the male connector, thereby preventing the cleaning solution from entering a lumen of the male connector, and wherein the center post is positioned to permit the cleaning solution expelled from the inner absorbent member to move past the center post towards the open first end of the housing and wet the outer surfaces of the male connector when the inner absorbent member is axially compressed.

In accordance with an embodiment of the present invention, wherein the housing, protrusion and shield includes a rigid thermoplastic polymer including at least one of polyester, polycarbonate, polypropylene, polyethylene, polyethylene terephthalate, or acrylonitrile butadiene styrene.

In accordance with an embodiment of the present invention, the cap further includes a cleaning solution absorbed by the inner absorbent support, wherein the cleaning solution includes Isopropyl Alcohol (IPA).

In accordance with an embodiment of the present invention, a method for attaching the cap to the male connector comprises inserting a distal end of a stem of the male connector through the open first end of the housing and into the second recess formed between the protrusion and the center post causing the distal end of the stem to contact the inner absorbent support and the center post to enter the central lumen in the stem of the male connector; and rotating the male connector relative to the housing causing the stem of the male connector to advance through the second recess axially compressing the inner absorbent support causing cleaning solution to be released into the second recess and thereby wetting, cleaning and disinfecting the distal end of a stem of the male connector outside of the central lumen.

In accordance with an embodiment of the present invention, inserting a distal end of a shield of the male connector through the open first end of the housing and into the first recess between the sidewall of the housing and the protrusion causing the distal end of the shield to contact the outer absorbent support; rotating the male connector relative to the housing causing the shield of the male connector to advance through the first recess between the sidewall of the housing and the protrusion causing the shield of the male connector to press against and compress the outer absorbent support and causing the cleaning solution to be expelled from the outer absorbent support and to move through the first recess thereby contacting, cleaning, and disinfecting the shield of the male connector.

In accordance with an embodiment of the present invention, a manufacturing method for a cap including forming the housing, protrusion and center post; inserting the inner absorbent support into the first recess between the sidewall of the housing and the protrusion; inserting the inner absorbent into the second recess between the protrusion and the center post; and affixing a protective cover to seal the open first end of the housing.

In accordance with an embodiment of the present invention, a cap is configured to engage a male connector including a central lumen and an annular shield, the cap including: a housing including an open first end, a second end, and a sidewall extending between the first end and the second end, and a protrusion including an open first end and a sidewall extending from the second end of the housing towards the open first end generally parallel to the sidewall of the housing configured to engage the male connector for retaining the male connector within the housing, and a center post extending from the center of the second end of the housing towards the center of the open first end of the housing configured to be inserted into and block the central lumen of male connector; an outer absorbent support disposed in the housing between the sidewall of the housing and the sidewall of the protrusion configured to contain a cleaning solution for cleaning and disinfecting portions of the male connector engaged to the cap including the annular shield of the male connector; and an inner absorbent support disposed between the protrusion and the center post configured to contain a cleaning solution for cleaning and disinfecting portions of the male connector engaged to the cap.

In accordance with an embodiment of the present invention, wherein the housing including the protrusion and the center post includes a rigid thermoplastic polymer, including at least one of polyester, polycarbonate, polypropylene, polyethylene, polyethylene terephthalate, or acrylonitrile butadiene styrene; and wherein the outer absorbent support and the inner absorbent support include sponges.

In accordance with an embodiment of the present invention, wherein the male connector includes a male luer connector including a luer stem including a tip defining the central lumen and the shield extends about the stem; and wherein the shield of the male luer connector includes threads on an inner surface of the shield configured to engage corresponding threads of a female luer connector for securing the male connector to the female luer connector.

In accordance with an embodiment of the present invention, wherein when the male connector is engaged to the cap: the shield of the male connector is disposed in a recess defined by the sidewall of the housing and the sidewall of the protrusion such that the shield contacts and axially compresses the outer absorbent support thereby releasing cleaning solution from the outer absorbent support that wets and thereby cleans and disinfects the shield of the male connector; the threads on the inner surface of the shield engage a thread extending from the sidewall of the protrusion thereby releasably connecting the male connector to the cap; the luer stem of the male connector is disposed in a recess defined by the sidewall of the protrusion and the center post such that the luer stem contacts and axially compresses the inner absorbent support thereby releasing cleaning solution from the inner absorbent support that wets and thereby cleans and disinfects the outside of the luer stem of the male connector including the tip of the luer stem; and the center post is disposed within the central lumen of male connector thereby blocking entry of the cleaning solution into the lumen.

In accordance with an embodiment of the present invention, a cap configured to engage a male connector includes a housing having an open first end, a second end, and a sidewall extending between the first end and the second end, and a protrusion including an open first end and a sidewall extending from the second end of the housing towards the open first end parallel to the sidewall of the housing, and a center post extending from the center of the second end of the housing towards the center of the open first end of the housing; an outer absorbent support disposed in the housing between the sidewall of the housing and the sidewall of the protrusion configured to contain a cleaning solution for cleaning and disinfecting portions of the male connector engaged to the cap; and an inner absorbent support disposed between the protrusion and the center post configured to contain a cleaning solution for cleaning and/or disinfecting portions of the male connector engaged to the cap.

In accordance with an embodiment of the present invention, the male connector includes a male luer connector.

In accordance with an embodiment of the present invention, wherein the male luer connector includes a luer stem defining a central lumen and a shield extending about the stem.

In accordance with an embodiment of the present invention, wherein the shield of the male luer connector includes threads on an inner surface of the shield configured to engage corresponding threads of a female luer connector for securing the male connector to the female luer connector.

In accordance with an embodiment of the present invention, wherein when the male luer connector is engaged to the cap, the luer stem of the male connector is inserted into a recess defined by the sidewall of the protrusion and the outside of the luer stem is in contact with the inner absorbent support and the inner surface of the shield of the male connector is in contact with the outer absorbent support.

In accordance with an embodiment of the present invention, wherein when the male connector is engaged to the cap, cleaning solution is exposed to the threads on the inner surface of the shield for cleaning and/or disinfecting the threads and cleaning solution is exposed to the outer surface of the luer stem for cleaning and/or disinfecting the luer stem.

In accordance with an embodiment of the present invention, wherein the cap is sized to receive male connectors having different thread configurations and dimensions.

In accordance with an embodiment of the present invention, wherein the housing includes a rigid thermoplastic polymer comprising at least one of polyester, polycarbonate, polypropylene, polyethylene, polyethylene terephthalate, or acrylonitrile butadiene styrene.

In accordance with an embodiment of the present invention, wherein the housing includes a plurality of ridges or ribs extending outwardly from an outer surface of the housing (for making the housing easier to grip).

In accordance with an embodiment of the present invention, wherein the inner absorbent support is axially compressible.

In accordance with an embodiment of the present invention, wherein the outer absorbent support is axially compressible.

In accordance with an embodiment of the present invention, wherein the outer absorbent support includes an annular member that is axially compressible and encloses the protrusion.

In accordance with an embodiment of the present invention, wherein the outer absorbent support includes a sponge.

In accordance with an embodiment of the present invention, wherein the protrusion includes an open first end, a second open end connected to the second end of the housing, and a sidewall extending between the first end and the second end.

In accordance with an embodiment of the present invention, wherein the second end of the protrusion is adhered to the second end of the housing, thereby securing the protrusion as part of the housing.

In accordance with an embodiment of the present invention, wherein the protrusion includes a rigid thermoplastic polymer, including at least one of polyester, polycarbonate, polypropylene, polyethylene, polyethylene terephthalate, or acrylonitrile butadiene styrene.

In accordance with an embodiment of the present invention, wherein the protrusion is formed from a same rigid thermoplastic polymer as the remainder of the housing.

In accordance with an embodiment of the present invention, wherein the inner absorbent support includes a sponge.

In accordance with an embodiment of the present invention, wherein the inner absorbent support includes a sponge including Isopropyl Alcohol (IPA).

In accordance with an embodiment of the present invention, wherein the inner absorbent support includes an open cell foam, such as a porous foam including a thermoplastic elastomer.

In accordance with an embodiment of the present invention, wherein insertion of the male connector into the recess formed by the protrusion causes the inner absorbent support to axially compress farther into the recess.

In accordance with an embodiment of the present invention, wherein the axial compression of the inner absorbent support expels the cleaning solution from the inner absorbent support causing the cleaning solution to move through an interior of the recess formed by the protrusion and contacting an outer surface of a stem of the male connector inserted in the recess.

In accordance with an embodiment of the present invention, wherein the axial compression of the inner absorbent support causes the cleaning solution of the inner absorbent support to pass through the protrusion and to be absorbed by the outer absorbent support.

In accordance with an embodiment of the present invention, further including the cleaning solution absorbed by the inner absorbent support.

In accordance with an embodiment of the present invention, wherein the cleaning solution includes or comprises common disinfectants known in the field such as Isopropyl Alcohol (IPA), Chlorhexidine Gluconate (CHG), chlorhexidine, povidone-iodine, and octenidine and any combination of them.

In accordance with an embodiment of the present invention, further including a protective cover over the open first end of the housing.

In accordance with an embodiment of the present invention, wherein the protective cover is attached to the housing by heat sealing.

In accordance with an embodiment of the present invention, a method for attaching the cap to the male connector includes inserting a distal end of a stem of the male connector through the open first end of the housing and the open end of the protrusion causing the distal end of the stem to contact the inner absorbent support and the distal end of the shield to contact the outer absorbent support; and rotating the male connector relative to the housing causing the stem of the male connector to advance through the recess formed by the protrusion and axially compressing the inner absorbent support.

In accordance with an embodiment of the present invention, further including continuing to rotate the male connector relative to the housing causing a shield of the male connector to press against and compress the outer absorbent support.

In accordance with an embodiment of the present invention, wherein advancing the stem of the male connector through the recess defined by the protrusion causes the cleaning solution to be expelled from the inner absorbent support and to move through the protrusion contacting an outer surface of the stem of the male connector.

In accordance with an embodiment of the present invention, wherein contact between the distal end of the stem and the center post seals a distal opening of the stem preventing the cleaning solution from passing into a lumen of the male connector.

In accordance with an embodiment of the present invention, wherein, upon axial compression of the inner absorbent support, the cleaning solution moves towards the open first end of the housing contacting an outer surface of the stem of the male connector.

In accordance with an embodiment of the present invention, a manufacturing method for the cap includes inserting the inner absorbent support into the socket; inserting the outer absorbent support into the housing; and forming the housing about an outer surface of the outer absorbent support by over-molding.

In accordance with an embodiment of the present invention, the housing includes a rigid thermoplastic polymer including at least one of polyester, polycarbonate, polypropylene, polyethylene, polyethylene terephthalate, or acrylonitrile butadiene styrene.

In accordance with an embodiment of the present invention, wherein forming the housing includes forming a bottom part of the housing from a rigid thermoplastic polymer and forming a top part of the housing from a thermoplastic elastomer.

### BRIEF DESCRIPTION OF THE DRAWINGS

FIG. 1A is a cross-sectional view of an exemplary male connector, as is known in the prior art.

FIG. 1B is an example of a closed female connector including a septum with a slit, as is known in the prior art.

FIG. 2A is a perspective view of a disinfecting cap for a male connector, according to an aspect of the present disclosure.

FIG. 2B is a cross-sectional view of the cap of FIG. 2A.

FIG. 2C is a perspective cross-sectional view of the cap of FIG. 2A.

FIG. 2D is an exploded view of the cap of FIG. 2A without a protective cover.

FIG. 3 is a perspective view of a cap of FIG. 2A without a protective cover, according to an aspect of the present disclosure.

FIG. 4A is a cross-sectional view of the cap of FIG. 2A and a male connector, prior to inserting the male connector into the cap.

FIG. 4B is a cross-sectional view of the cap of FIG. 2A with the male connector fully inserted into the cap.

FIG. 5 is a flow chart showing a method for attaching the disinfecting cap to a male connector, according to an aspect of the present disclosure.

FIG. 6 is a cross-sectional view of the cap of FIG. 2A with the male connector fully inserted into the cap.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. The term “proximal” refers to a portion of a device or part that is grasped by a user or connected to another device or part. The term “distal” refers to a portion of a device or part that is opposite the proximal portion (i.e., farthest away from the portion that is grasped by a user or connected to another part). For example, for an implantable medical device, such as a catheter, a proximal portion can refer to the portion of the catheter that remains outside of a patient's body and is manipulated by a user. The distal portion of the catheter can be the portion that is inserted into the vasculature of the patient. For an object or part, such as an elongated member, the proximal end can be the end that is connected to another object or part. The distal end of the elongated member is opposite the proximal end and can be free from connections to other objects or parts. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

The present disclosure is directed to a disinfecting cap 10 configured to be connected to a male connector 110, such as a male connector of an access hub, port, or valve for a VAD, to prevent the connector, port, or VAD from being contaminated by, for example, microbes, debris, or other contaminants. For example, the disinfecting cap 10 can be a male disinfecting cap for disinfecting ISO594-2 or ISO80369-7 type of female threaded fluid luer connectors. In some examples, the cap 10 can be configured to clean or disinfect portions of the connector 110 or port, ensuring that the connector 110 or port remains sterile prior to use. The cap 10 can be configured to remain in place on a connector 110 or port for at least seven days, which is a maximum time of recommended use permitted by many medical facility sterile practice guidelines.

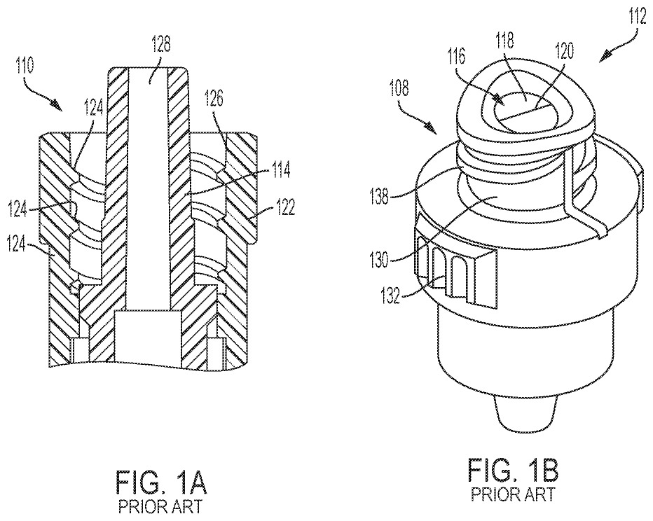

The cap 10 is a disinfecting cap for male connectors configured to engage with or be connected to different sizes, configurations, and/or types of male connectors 110. As used herein, a “male connector” refers to a connector 110 comprising an elongated member, such as a tubular member or stem 114, configured to be inserted in a tube or opening having an inner diameter that is larger than an outermost diameter of the male connector 110. An exemplary male connector 110 is shown in FIG. 1A. The male connector 110 includes a lumen 128 and is configured to be connected to a hub, port, or another portion of a VAD comprising a female connector. As used herein, the “female connector” refers to a connector 112 comprising an opening or port 116 that is configured to receive an elongated member or tubular member of another object or device in order to connect the object or device to the female connector 112. The female connector 112 can comprise an elongated distal end portion 108 with a cover or septum 118 over the opening 116. An exemplary female connector 112 including a septum 118 with a slit 120 is shown in FIG. 1B.

In some examples, the cap 10 can be configured to be connected to or engage different types (e.g., different sizes and shapes) of male luer connectors. For example, the cap 10 can be sized to receive different types of male luer connector 110 having an outer diameter of from about 8.0 mm to about 12.0 mm. As used herein, a “luer connector” refers to a connector that includes a tapered portion (i.e., a luer taper) for creating a friction engagement between a tapered stem 114 or elongated member of a male luer connector 110 and a tapered cavity. For example, the male luer connector 110 can include a tapered stem 114 or elongated member having a tapered outer surface. A female luer connector 112 configured to engage the male luer connector 110 can include a tapered cavity configured to receive and engage the tapered stem 114 or elongated member to connect the male luer connector 110 to the female luer connector 112.

In some examples, the male connectors 110 can also include engaging structures, such as threads, for drawing the male connectors 110 into or against a female port or hub. For example, as shown in FIG. 1A, the male luer connector 110 can include an annular shield 122 extending about the tapered stem 114 or elongated member. The annular shield 122 can include threads 124 on an inner surface 126 of the shield 122 configured to engage corresponding threads 138 on an outer surface 130 of the female luer connector 112. Specifically, as shown in FIG. 1B, the female luer connector 112 can include threads 138 extending from the outer surface 130 positioned to engage the threads 124 on the inner surface 126 of the annular shield 122 of the male luer connector 110. Twisting the female connector 112 relative to the male connector 110 causes the corresponding threads 124, 138 to engage, which draws the connectors 110, 112 together, such that the tapered stem 114 or elongated member of the male luer connector 110 moves through the opening 116 of the female connector 112. In some examples, the female connector 112 can also include vertical ribs 132 near a proximal end of the female connector 112, which can be used to manipulate the female connector 112 making it easier to twist the female connector 112 relative to another connector or device.

There are numerous commercially available medical devices, such as hubs, ports, and valves, which include different variations of male connectors 110, such as male luer connectors. As described in further detail herein, in some examples, the cap 10 of the present disclosure includes a flexible housing that can stretch, bend, or otherwise deform in order to engage and securely connect to different types and sizes of male connectors 110. For example, the cap 10 can be configured to attach to a male Luer-Lok™ connector by Becton Dickinson and Company. The cap 10 can also be configured to cover different connector designs including, without limitation, the BD Q-Syte™, BD MaxZero™, BD MaxPlus™, and SmartSite™ needle free connectors by Becton Dickinson and Company. The cap 10 can also be configured to be connected to male connectors by other manufactures including, without limitation, MicroClave® connectors (ICU Medical Inc.) and Ultrasite® connectors (B. Braun Medical Inc.). In other examples, the cap 10 can be configured to connect to one or more of the following commercially available male connectors: Kendall 2001NP; BD MP5303-C; ICU Med 12664-28; RyMed RYM-5307HPU; B. Braun 470108; Baxter 2C8537; Kawasumi IV-0094; Zyno B2-70071-D; B. Braun 470124; Baxter 2C7462; and Smith's Medical 536035.

### Male Disinfection Cap

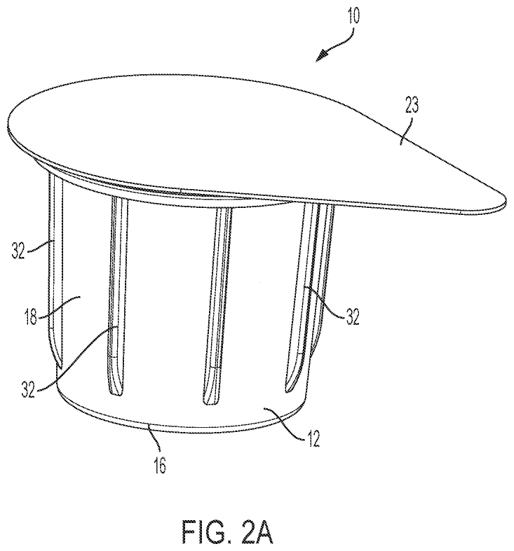

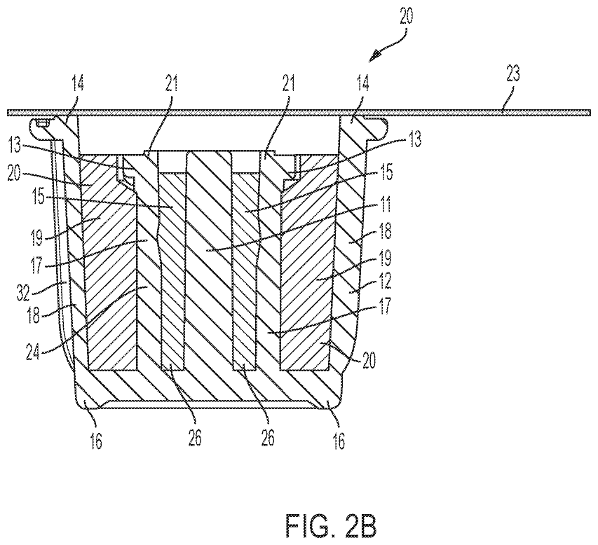

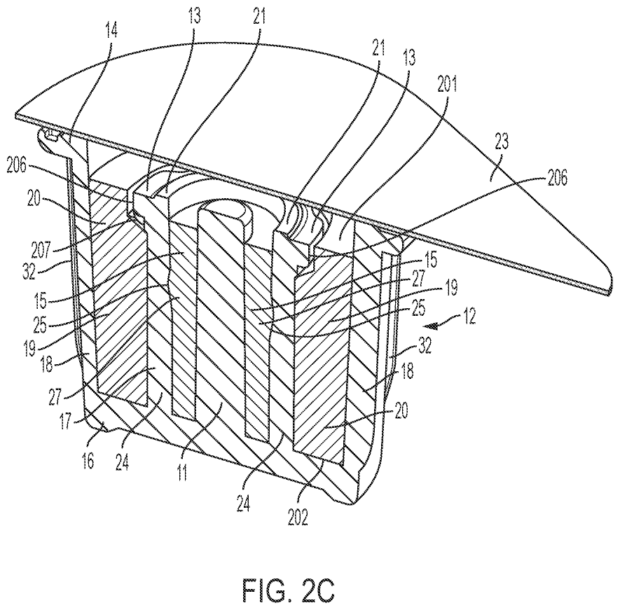

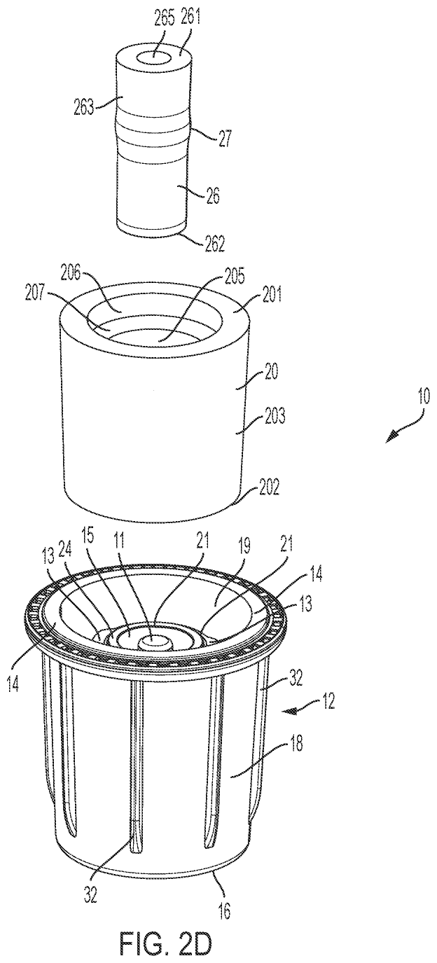

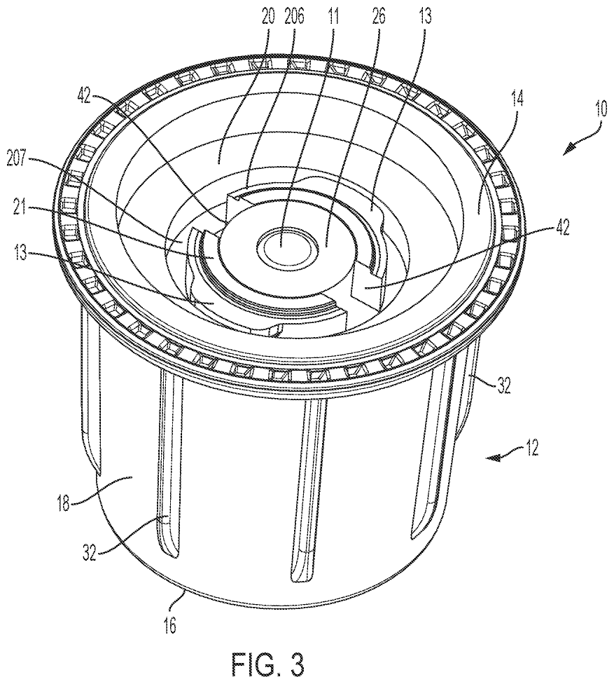

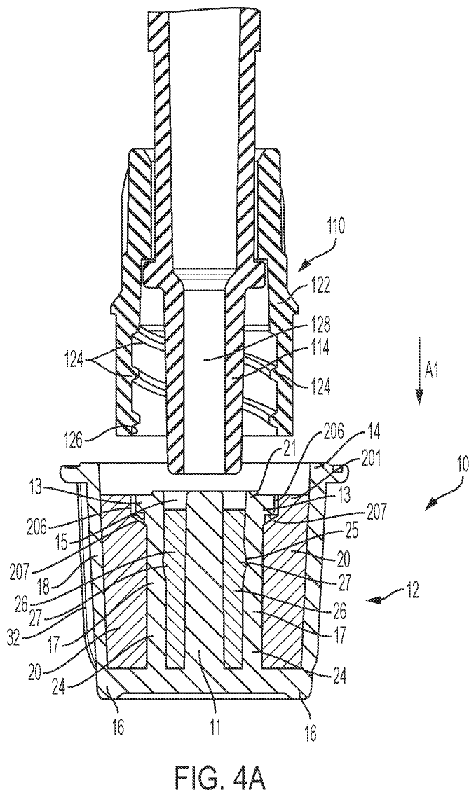

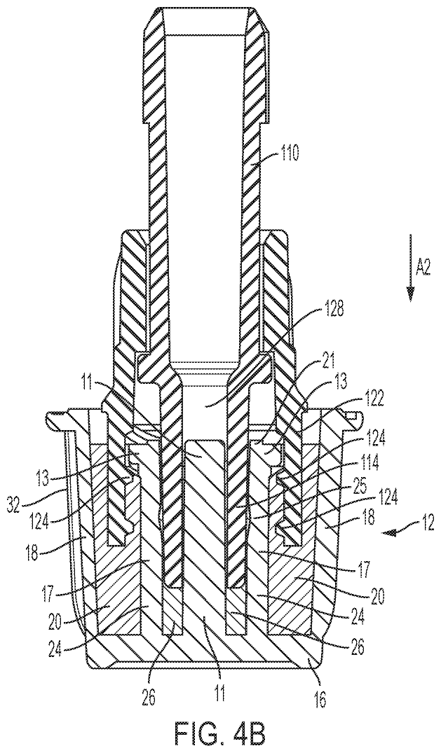

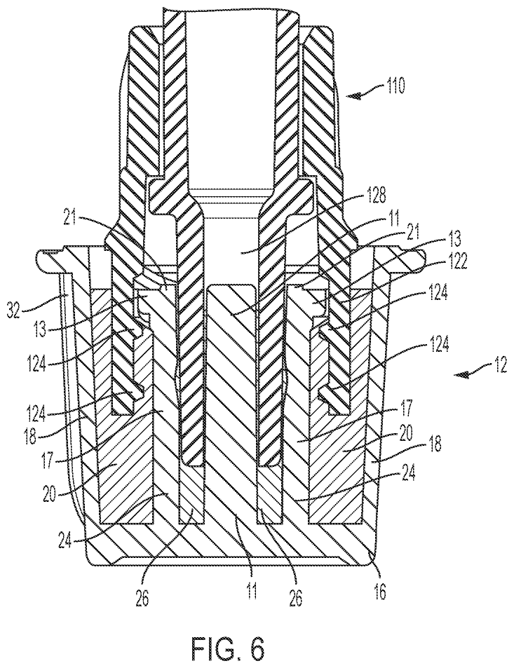

FIGS. 2A-4B and 6 illustrate portions of an exemplary disinfecting cap 10 configured to engage and/or to be connected to male connectors 110 with different shapes, sizes, dimensions, or configurations. Specifically, FIG. 2A is a perspective view of the cap 10 prior to use with a protective cover adhered over an open top or first end of the cap 10. FIG. 2B is a cross-sectional view of the cap 10 with the protective cover in place. FIG. 2C is a perspective cross-sectional view of the cap 10 with the protective cover in place. FIG. 2D is an exploded view of the cap 10 without the protective cover. FIG. 3 is a perspective view of a housing of the cap 10 with the protective cover removed showing an open top or first end of the housing. FIG. 4A is a cross-sectional view of the cap 10 and a male connector 110 before it is inserted into the cap 10. FIG. 4B is a cross-sectional view showing the male connector 110 fully inserted into the disinfecting cap 10. FIG. 6 is a close-up cross-sectional view showing the male connector 110 fully inserted into the disinfecting cap 10.

In some examples, the cap 10 can be provided as a single pre-packaged cap or cap assembly, such as the packaged cap shown in FIG. 2A. Further, as described in detail herein, the cap 10 includes components, such as sponges, and/or cleaning or disinfecting solutions, for cleaning, scrubbing, and disinfecting portions of the male connectors 110 inserted into and mounted to the cap 10.

As shown in FIGS. 2A-4C, the cap 10 comprises a housing 12 comprising an open first end or top 14, a second end or bottom 16, and a sidewall 18 extending between the top 14 and the bottom 16 forming a first recess 19. The housing 12 further comprises a protrusion 24 disposed in the first recess 19 of the housing 12 comprising an open first end 21 and a sidewall 17 extending between the open first end 21 and the bottom 16 forming a second recess 15. The protrusion 24 is connected, molded or attached to the second end or bottom 16 of the housing 12 and is configured to engage the male connector 110 for retaining the male connector 110 within the housing 12. The housing 12 also comprises a center post 11 extending from the center of the second end or bottom 16 of the housing 12 into the center of the second recess 15 formed defined by the protrusion 24. The cap 10 further comprises an outer absorbent support 20 disposed in the first recess 19 of the housing 12. The outer absorbent support 20 can be a tubular member defining a central recess 22 or cavity sized to receive portions of the male connector 110. The cap 10 further comprises an inner absorbent support 26 disposed in the second recess 15 defined by the protrusion 24 of the housing 12. For example, the stem 114 of the male connector 110 can be inserted into the second recess 15 formed between the protrusion 24 and the center post 11 and threads 124 on the inner surface 126 of the shield 122 can engage corresponding threaded structures or a luer lug 13 on the outer surface of the protrusion 24 for securing the male connector 110 to the protrusion 24.

The cap 10 also includes cleaning parts, such as sponges, brushes, or porous members, disposed in the first recess 19 defined by the housing and the second recess 15 defined by the protrusion 24, for cleaning distal portions of the male connector 110. For example, the cap 10 can comprise an inner absorbent support 26 disposed in the second recess 15 defined by the protrusion 24 and an outer absorbent support 20 disposed in the first recess 19 defined by the housing 12. The inner absorbent support 26 may comprise a tubular structure with an open first end 261, an open second end 262, and a sidewall 263 connecting said first end and said second end defining an opening 265 along the central axis of the inner absorbent support 26 as best shown in FIG. 2D. The inner absorbent support 26 may also comprise a bump or ridge 27 disposed around the outside surface of the inner absorbent support 26 as shown in FIG. 2D to better provide a fiction fit with the protrusion 24 of the cap 12 as shown in FIG. 2C and FIGS. 4A-4B and specifically with a recess 25 within the inner sidewall of the protrusion 24. The outer absorbent support 20 may comprise a tubular structure with an open first end 201, an open second end 202, and a sidewall 203 connecting said first end and said second end defining an opening 205 along the central axis of the outer absorbent support 20 as best shown in FIG. 2D. The outer absorbent support 20 may comprise a recess 206 proximate the open first end 201 that is of a greater diameter than the opening 205 and that creates an annular inner surface 207 within the opening 205 of the outer absorbent support 20. The recess 206 and the annular inner surface 207 can be configured to accommodate or surround a thread or luer lug 13 on the protrusion 24 as shown FIGS. 2C and 4A for example. The inner absorbent support 26 and the outer absorbent support 20 are configured to contain a cleaning solution for cleaning and/or disinfecting portions of the male connector 110 engaged to the cap 10 including the tip of the stem of the male connector.

The outer absorbent support 20, inner absorbent support 26, and other parts of the cap 10 of the present disclosure are configured to cause the cleaning solution to contact multiple areas of the male connector 110 ensuring that the multiple areas of the male connector 110 remain sterile and free from microbes, contaminants, particles, and other debris. In particular, the cap 10 is configured to ensure that cleaning solution contacts not only a distal end or tip of the stem 114 of the male connector 110, but also outer surfaces of the stem 114, as well as inner and outer surfaces of the annular shield 122. Importantly, the cleaning solution should contact both roots and crests of the threads 124 on the inner surface 126 of the shield 122 to ensure that the threads 124 remain clean, sterile, and ready for use.

In some examples, the housing 12 is a molded part formed by injection molding or other common molding processes. In some examples, the housing 12 can be formed from a rigid thermoplastic polymer material, such as polyester, polycarbonate, polypropylene, polyethylene, polyethylene terephthalate, or acrylonitrile butadiene styrene. Further, the housing 12 can be formed from a durable material, such as a material having a shore hardness D value of less than or equal to 95 (Shore D). Alternatively, the housing 12 can be formed from a more flexible material, such as a material having a shore hardness A value less than or equal to 130 (Shore A). Beneficially, a flexible housing 12 or housing 12 including flexible portions can be capable of stretching, bending, or otherwise deforming so that the cap 10 is capable of engaging male connectors 110 of different sizes and shapes.

In some examples, interior portions of the housing 12 can be shaped to retain the outer absorbent support 20 which can be annular in shape within the housing 12 as shown in FIGS. 2B-2C. The interface or friction engagement between the sidewall 18 of the housing 12, the sidewall 17 of the protrusion 24, and the outer absorbent support 20 can be configured to ensure that the outer absorbent support 20 remains seated in the first recess 19 of housing 12. The interface or friction engagement between the sidewall 17 of the protrusion 24, the center post 11, and the inner absorbent support 26 can be configured to ensure that the inner absorbent support 26 remains seated in the second recess 15 defined by the protrusion 24 in the housing 12. In particular the interface or friction engagement can ensure that the outer absorbent support 20 does not lift away from the housing 12 when the male connector 110 is inserted into or removed from the housing 12.

In some examples, the housing 12 further comprises protrusions, such as axially extending ribs or ridges 32, extending outward from an outer surface of the sidewall 18 of the housing 12. The axial ridges 32 can be provided to increase rigidity of the housing 12 compared to if ridges 32 were not present. Also, the ridges 32 can make the housing 12 easier to grasp and manipulate improving usability of the cap 10 and, for example, making it less likely that the practitioner will drop or mishandle the cap 10 during use.

As shown most clearly in FIGS. 2B and 2C, the cap 10 further comprises the outer absorbent support 20 disposed within the first recess 19 of the housing 12, which can also be referred to as an absorber, absorbent member, cushion, padding, or sponge. As described in further detail herein, the outer absorbent support 20 is a flexible, deformable, or compressible structure configured to compress axially when the male connector 110 is inserted into the housing 12. In particular, the outer absorbent support 20 is configured to compress so that the annular shield 122 of the male connector 110 can be inserted farther into the housing 12 allowing threads 124 of the shield 122 to come into contact with an engage the luer lugs 13 of the protrusion 24. In some examples, the outer absorbent support 20 can be an annular or tubular structure defining the first recess 19 having an outer diameter that substantially matches (e.g., is the same as or within about 5% or less of) an inner diameter of the sidewall 18 of the housing 12. In some examples, the recess 19 can be an irregular shape, such as a recess or cavity formed from different sized connected cylindrical and/or elongated cavities.

The outer absorbent support 20 can be formed from a flexible and/or compressible material that deforms and presses against portions of the male connector 110 as the male connector 110 is inserted into the housing 12. For example, the outer absorbent support 20 can be configured to axially compress as the male connector 110 is inserted to the housing 12. Also, the outer absorbent support 20 can be configured to deform or bulge radially inwardly pressing against surfaces of the male connector 110. For example, the outer absorbent support 20 can press against a distal end of the annular shield 122 and against an outer surface of the annular shield 122 to seal portions of the male connector 110, which prevents contamination of inner spaces enclosed by the annular shield 122. Also, the outer absorbent support 20 can be configured to press against the outer surface of the annular shield 112 pushing into any gaps, slots, cut-away portions, channels, cavities, grooves, recessed areas, or any other spaces on the outer surface of the annular shield 122, which eliminates any spaces where contaminants, such as microbes, dirt, particles, or other debris, may collect. Accordingly, the deformable outer absorbent support 20 contributes to forming a tight and secure connection between surfaces of the male connector 110 and interior component of the cap 10, thereby contributing to the cleaning, disinfecting, and contamination avoiding features of the male connector 110.

In some examples, the outer absorbent support 20 comprises a thermoplastic elastomer, such as polypropylene, polyethylene, or synthetic or natural rubber (e.g., isoprene). The outer absorbent support 20 can also comprise a porous foam (e.g., an open cell foam) or sponge capable of absorbing the cleaning or disinfecting solution, such as a foam or sponge comprising polyurethane. In other examples, the foam material can be a Plastazote® foam, which is an engineered polymer foam by Zotefoams PCL.

The cap 10 further comprises the protrusion 24, which engages the male connector 110, thereby securing the male connector 110 within the housing 12. As shown in FIGS. 2B and 2C, the protrusion 24 can be a tubular member secured or adhered to the bottom 16 housing 12 comprising an open first end or top 21 and a sidewall 17 extending between the top 21 and the bottom 16. For example, as shown in FIG. 2B, the protrusion 24 can be shaped as an open annular cylinder closed at the bottom by the inner surface of the bottom 16 of the housing 12. The protrusion 24 can be entirely enclosed within the housing 12 meaning, for example, that the open top 21 of the protrusion 24 is recessed relative to the open top 14 of the housing 12 as shown in FIGS. 2C-2B.

The protrusion 24 generally and the center post 11 are formed from or comprise a rigid thermoplastic polymer material, such as the same material used to form the remainder of the housing 12. For example, the protrusion 24 and center post 11 can be formed from a rigid polymer material, such as polyester, polycarbonate, polypropylene, polyethylene, polyethylene terephthalate, or acrylonitrile butadiene styrene.

In some examples, the protrusion 24 further comprises a thread or luer lug 13 extending radially outward from the sidewall 17 of the protrusion 24. The thread 13 can be configured to engage threads 124 on an inner surface 126 of the annular shield 122 of the male connector 110 for securing the male connector 110 to the protrusion 24. Accordingly, the male connector 110 can be securely retained within the housing 12 by the threaded engagement preventing the cap 10 from being mistakenly removed from the male connector 110 at unexpected or inappropriate times.

In some examples, the protrusion 24 further comprises one or more openings in the sidewall 17 of the protrusion 24 for permitting liquid, such as cleaning solution, to pass from an interior second recess 15 defined by the protrusion 24 and the inner absorbent support 26 to the outer absorbent support 20 surrounding the protrusion 24. For example, as shown in FIG. 3, the opening can be a slot 42 extending axially from the open top 21 of the protrusion 24. In some examples, as shown in the figures, the protrusion 24 can include opposing slots 42 in the sidewall 17 of the protrusion 24 on opposite sides of the protrusion 24 for providing cleaning solution to different areas or regions of the outer absorbent support 20. In other examples, the openings can be holes (e.g., square, rectangular, circular, or elliptical holes), slits, perforations, or any other openings extending through the sidewall 17 of the protrusion 24 for permitting the cleaning solution to pass from the interior second recess 15 formed by the protrusion 24 to the outer absorbent support 20.

The cap 10 further comprises the inner absorbent support 26 disposed within second recess 15 formed by the protrusion 24. As previously described, the inner absorbent support 26 contains the cleaning solution for cleaning and/or disinfecting portions of the male connector 110 engaged to the cap 10. As shown in FIG. 2B, the inner absorbent support 26 can be disposed and held within a cylindrical interior recess or cavity 15 defined by the protrusion 24 and the center post 11. For example, the inner absorbent support 26 can be held in place within the second recess 15 formed by the protrusion 24 and the center post 11 by a conventional adhesive or mechanical fastener. In other examples, the inner absorbent support 26 can be held in place in the second recess 15 formed by the protrusion 24 and the center post 11 by friction between an inner surface of the sidewall 17 of the protrusion 24 and an outer surface of the inner absorbent support 26 and the outer surface of the center post 11 and the inner surface of the inner absorbent support 26. The inner absorbent support 26 can be tubular in shape in some examples.

The inner absorbent support 26 can be formed from the same material as the outer absorbent support 20, specifically from a material that is capable of absorbing the cleaning solution and expelling the cleaning solution from the inner absorbent support 26 when compressed. For example, the inner absorbent support 26 can be formed from a thermoplastic elastomer, such as polypropylene, polyethylene, or synthetic or natural rubber (e.g., isoprene). The inner absorbent support 26 can also include a porous foam (e.g., an open cell foam) or sponge capable of absorbing the cleaning or disinfecting solution, such as a foam or sponge comprising polyurethane. In other examples, the foam material can be a Plastazote® foam, which is an engineered polymer foam by Zotefoams PCL.

In some examples, the inner absorbent support 26 can be provided (i.e., presoaked) with the cleaning or disinfecting solution. For example, the cleaning or disinfecting solution can be an antimicrobial, anti-fungal, antibacterial, or antiviral solution that cleans and sterilizes surfaces of the male connector 110. In some examples, the cleaning solution can be comprised of isopropyl alcohol (IPA).

The inner absorbent support 26 can be configured to axially compress as the stem 114 of the male connector 110 is inserted into and moves through the interior of the second recess 15 formed by the protrusion 24 and the center post 11. Axial compression of the inner absorbent support 26 expels the cleaning solution from the inner absorbent support 26. The expelled cleaning solution moves through interior of the second recess 15 formed by the protrusion 24 and the center post 11 towards the open top 21 of the protrusion 24 contacting an outer surface of the stem 114 of the male connector 110 inserted into the second recess 15. Once expelled from the inner absorbent support 26, the cleaning solution can also pass through openings, such as the slots 42, of the protrusion 24. After passing through the slots 42, the cleaning solution comes into contact with other parts of the male connector 110, such as the threads 124, inner surface 126, and/or outer surface of the annular shield 122. The cleaning solution can also be absorbed by the outer absorbent support 20, such that cleaning solution remains in proximity to surfaces of the annular shield 122 for cleaning and disinfecting these surfaces.

In some examples, the cap 10 further comprises a center post 11 positioned in the center of the housing 12 within the second recess 15 defined by the protrusion 24. The center post 11 can prevent fluids, such as cleaning or disinfecting solution absorbed by the inner absorbent support 26, from flowing into the lumen 128 enclosed by the stem 114 of the male luer connector 110.

The center post 11 is sized and positioned so that cleaning solution expelled from the inner absorbent member 26 can move past the center post 11 towards the open top 21 of the protrusion 24 and/or the top 14 of the housing 12. As previously discussed, as the cleaning solution moves through the interior second recess 15 defined by the protrusion 24, the cleaning solution contacts the outer surface of the stem 114 for cleaning and disinfecting the outer surface. In order for the cleaning solution to move past the center post 11, the outer diameter of the center post 11 can be smaller than an inner diameter of the protrusion 24 so that the center post 11 does not prevent the cleaning solution from moving towards the top open end 21 of the protrusion 24 and coming into contact with the outer surface of the stem 114.

In some examples, as shown in FIGS. 2A-2C, the cap 10 can further comprise a removable and/or disposable protective cover 23 positioned over the open top 14 of the housing 12. The protective cover 23 can be provided to protect components and portions of the cap 10, such as the housing 12, outer absorbent support 20, protrusion 24, inner absorbent support 26, and center post 11, during transport and storage, as well as to prevent contamination and to prevent the cleaning or disinfecting solution from evaporating prior to use. The protective cover 23 can comprise a sheet, such as a polymer film, with adhesive on a first side of the sheet for removably mounting the protective cover 23 to the open top 14 of the housing 12. Alternatively, the protective cover 23 can be removably mounted to the open top 14 of the housing 12 by heat sealing. The protective cover 23 can be formed from a material that is impervious or substantially impervious to air, so that the cleaning solution absorbed by the inner absorbent support 26 and outer absorbent support 20 does not evaporate or dry-out prior to use of the cap 10. Accordingly, the protective cover 23 can increase a shelf life of the cap 10, as well as prevent microbes and other debris from collecting in the cap 10 prior to use.

In the examples set forth in this disclosure, the center post 11 may prevent entry of IPA inside the lumen 128 of the male connector 110. The outer absorbent support or sponge 20 disinfects the thread portion 124 external wall of the shield 122 of the male connector 110. The inner absorbent support or sponge 26 disinfects the flat face or tip of the stem 114 of the male connector 110 outside of the lumen 128.

### Method for Attaching a Connector to the Cap

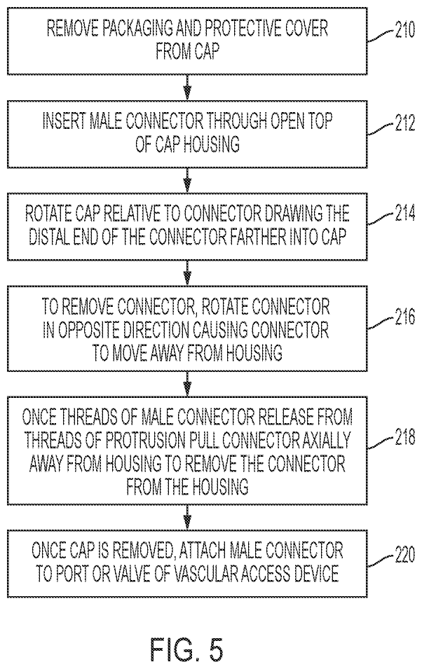

As previously described, the cap 10 of the present disclosure is a disinfecting cap 10 configured to be connected to various types and sizes of male connector 110. FIGS. 4A-4B and FIG. 6 show the cap 10 being connected to a male connector 110. FIG. 5 is a flow chart showing a method for connecting and/or disconnecting the disinfecting cap 10 from the male connector 110.

As shown in FIG. 5, at step 210, in order to connect the cap 10 to a male connector 110, the practitioner first removes any packaging from the cap 10 and removes the protective cover 23 from the open top 14 of the housing 12.

At step 212, once the packaging and protective cover 23 are removed, the practitioner moves the connector 110 towards the open top 14 of the housing 12 in a direction of arrow A1 (shown in FIG. 4A). Specifically, the stem 114 of the male connector 110 is inserted through the open top 14 of the housing 12 and through the open top 21 of the protrusion 24 causing the distal end of the stem 114 of the male connector 110 to contact the center post 11 disposed within the second recess 15 formed by the protrusion 24, as shown in FIG. 4B. Also, contact between the distal end of the stem 114 and the center post 11 can seal off the lumen 128 extending through the stem 114 preventing cleaning solution or other fluids from passing into the lumen 128 of the male connector 110. Specifically, as shown ion FIG. 4B, the center post 11 extends into the lumen 128 through the distal end of the stem 114 thereby mechanically sealing off the lumen 128 from contact with the inner absorbent support 26 and disinfectant and/or cleaning solution that the support may hold. Contact between the distal end of the stem 114 and the center post 11 can also mechanically remove particles, such as microbes, dirt, and other debris, from the male connector 110. Moving the male connector 110 towards the cap 10 also brings the annular shield 122 of the male connector 110 into contact with the protrusion 24. Specifically, as shown in FIG. 4B, the threads 124 of the shield 122 are brought into contact with the thread or luer lug 13 on the outer surface of the protrusion 24.

At step 214, in order to move the male connector 110 farther into the housing 12 of the cap 10, the practitioner next rotates the male connector 110 relative to the cap 10. Rotation of the male connector 110 relative to the housing 12 and protrusion 24 draws the male connector 110 farther into the cap 10, as shown by arrow A2 in FIG. 4B, causing the distal end of the stem 114 to move through the interior of the open top 13 and second recess 15 formed by the protrusion 24. As the stem 114 moves through the second recess 15 formed by the protrusion 24, the inner absorbent support 26 axially compresses expelling the cleaning solution from the inner absorbent support 26 into the interior second recess 15 formed by the protrusion 24. The cleaning solution moves from the inner absorbent support 26 towards the open top 21 of the protrusion 24. As the cleaning solution is pushed through the second recess 15 formed by the protrusion 24, the cleaning solution contacts the outer surface of the stem 114 cleaning and disinfecting the outer surface of the stem 114. The cleaning solution then passes through the slots 42 in the sidewall 17 of the protrusion 24 and is absorbed by the outer absorbent support 20.

Rotation of the male connector 110 relative to the housing 12 also causes the annular shield 122 to move into the top section of the first recess 19 defined by the sidewall 18 of the housing 12. Continued movement of the annular shield 122 into the housing 12 causes the shield 122 to press against the outer absorbent support 20, which compresses the outer absorbent support 20. As previously described, the outer absorbent support 20 can also bulge radially inward pushing into gaps, slots, cut-away portions, channels, cavities, grooves, or any other spaces on the outer surface of the annular shield 122, which eliminates gaps or open spaces where contaminants, such as microbes, dirt, or other debris, may collect. As previously described, cleaning solution from the inner absorbent support 26 can also pass towards the threads 124 of the annular shield 122 through the slots 42 of the protrusion 24 for cleaning and disinfecting the threads 124 of the annular shield 122. The cleaning solution can also be absorbed by the outer absorbent support 20, such that cleaning solution remains in close proximity to surfaces of the annular shield 122, thereby protecting the inner and outer surfaces of the annular shield 122 from contamination.

At step 216, in order to remove the cap 10 from the male connector 110, the practitioner first rotates the male connector 110 relative to the housing 12 and protrusion 24 causing the threads 124 of the annular shield 122 to back off of the thread 13 on the outer surface of the protrusion 24. The rotation of the male connector 110 relative to the protrusion 24 causes the male connector 110 to begin to move axially out of the housing 12.

At step 218, once the threads 124 of the male connector 110 are released from the thread 13 of the protrusion 24, the practitioner can pull the male connector 110 away from the housing 12, thereby releasing the male connector 110 from the cap 10. For example, the practitioner can grasp the housing 12 of the cap 10 with one hand and the male connector 110 with the other hand. The practitioner then pulls the male connector 110 away from the cap 10 to remove the male connector 110 from the cap 10. Once removed, the cap 10 can be discarded, as it is often a disposable single-use product.

Once the male connector 110 is fully removed from the housing 12, at step 220, the male connector 110 can be connected to a VAD. For example, the male connector 110 can be attached or inserted into a female hub, port, or valve of the VAD forming a needleless fluid-tight connection between the male connector 110 and a fluid path, channel, or lumen 128 of the VAD.

While examples of the disinfecting cap 10 and methods of use of the present disclosure are shown in the accompanying figures and described hereinabove in detail, other examples will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

The invention claimed is:

1. A cap configured to engage a male connector, the cap comprising:
a housing comprising an open first end, a closed second end, and a sidewall extending between the first end and the second end;
a protrusion extending from the closed second end of the housing towards the open first end of the housing configured to engage the male connector for retaining the male connector within the housing and defining a first recess between the sidewall of the housing and the protrusion;
a center post extending from the closed second end of the housing towards the open first end of the housing configured to engage the male connector and defining a second recess between the protrusion and the center post;
an outer absorbent support disposed in the first recess configured to contain a cleaning solution for cleaning and disinfecting portions of the male connector engaged to the cap; and
an inner absorbent support disposed in the second recess configured to contain a cleaning solution for cleaning and disinfecting portions of the male connector engaged to the cap.

2. The cap of claim 1, wherein the male connector comprises a luer stem comprising a tip and defining a central lumen and an annular shield extending about the stem,
wherein the shield of the male luer connector comprises threads on an inner surface of the shield configured to engage corresponding threads of a female luer connector for securing the male connector to the female luer connector, and
wherein the protrusion comprises an outer surface and threads on the outer surface of the protrusion configured to engage the threads on the inner surface of the shield of the male connector thereby releasably securing the male connector to the cap.

3. The cap of claim 2, wherein, when the male connector is engaged to the cap, the luer stem of the male connector is inserted into the second recess and in contact with the inner absorbent support, the center post is inserted into the central lumen of the male connector, and the annular shield of the male connector is in contact with the outer absorbent support.

4. The cap of claim 3, wherein, when the male connector is engaged to the cap, cleaning solution is exposed to the threads on the inner surface of the annular shield for cleaning and disinfecting the threads, cleaning solution is exposed to the outer surface of the luer stem for cleaning and disinfecting the outer surface of the luer stem; and the center post blocks cleaning solution from entering the central lumen of the male connector.

5. The cap of claim 1, further comprising a protective cover configured to be affixed to the open first end of the housing to prevent exposure of the protrusion, center post, outer absorbent support, and inner absorbent support to contamination prior to use of the cap.

6. The cap of claim 1, wherein the protrusion further comprises:
a thread extending radially outward from the sidewall of the protrusion, the thread configured to engage threads on an inner surface of a shield of the male connector.

7. The cap of claim 6, wherein the engagement between the thread of the protrusion and the threads of the shield of the male connector rotatably secures the male connector within the housing.

8. The cap of claim 7, wherein the sidewall of the protrusion comprises at least one opening for permitting the cleaning solution to pass from the inner absorbent support through the protrusion to the outer absorbent support.

9. The cap of claim 1, wherein insertion of the male connector into the second recess formed between the protrusion and the center post causes the inner absorbent support to axially compress farther into the second recess, and
wherein the axial compression of the inner absorbent support expels the cleaning solution from the inner absorbent support causing the cleaning solution to move through the second recess contacting an outer surface of a stem of the male connector inserted in the second recess including the tip of the stem of the male connector.

10. The cap of claim 1, wherein insertion of the male connector into the first recess formed between the housing and the protrusion causes the outer absorbent support to axially compress farther into the first recess, and
wherein the axial compression of the outer absorbent support expels the cleaning solution from the outer absorbent support causing the cleaning solution to move through the first recess contacting a shield of the male connector inserted in the first recess.

11. The cap of claim 1, wherein the central post is configured to seal an open end of the male connector, thereby preventing the cleaning solution from entering a lumen of the male connector, and
wherein the center post is positioned to permit the cleaning solution expelled from the inner absorbent member to move past the center post towards the open first end of the housing and wet the outer surfaces of the male connector when the inner absorbent member is axially compressed.

12. The cap of claim 1, wherein the housing, protrusion and shield comprise a rigid thermoplastic polymer comprising at least one of polyester, polycarbonate, polypropylene, polyethylene, polyethylene terephthalate, or acrylonitrile butadiene styrene.

13. The cap of claim 1, further comprising the cleaning solution absorbed by the inner absorbent support, wherein the cleaning solution comprises at least one of Isopropyl Alcohol (IPA), Chlorhexidine Gluconate (CHG), chlorhexidine, povidone-iodine, or octenidine.

14. A method for attaching the cap of claim 2 to the male connector, the method comprising:
inserting a distal end of a stem of the male connector through the open first end of the housing and into the second recess formed between the protrusion and the center post causing the distal end of the stem to contact the inner absorbent support and the center post to enter the central lumen in the stem of the male connector; and
rotating the male connector relative to the housing causing the stem of the male connector to advance through the second recess axially compressing the inner absorbent support causing cleaning solution to be released into the second recess and thereby wetting, cleaning and disinfecting the distal end of a stem of the male connector outside of the central lumen.

15. The method of claim 14 further comprising:
inserting a distal end of a shield of the male connector through the open first end of the housing and into the first recess between the sidewall of the housing and the protrusion causing the distal end of the shield to contact the outer absorbent support;
rotating the male connector relative to the housing causing the shield of the male connector to advance through the first recess between the sidewall of the housing and the protrusion causing the shield of the male connector to press against and compress the outer absorbent support and causing the cleaning solution to be expelled from the outer absorbent support and to move through the first recess thereby contacting, cleaning, and disinfecting the shield of the male connector.

16. A manufacturing method for the cap of claim 1, the method comprising:
forming the housing, protrusion and center post;
inserting the inner absorbent support into the first recess between the sidewall of the housing and the protrusion;
inserting the inner absorbent into the second recess between the protrusion and the center post; and
affixing a protective cover to seal the open first end of the housing.

17. A cap configured to engage a male connector comprising a central lumen and an annular shield, the cap comprising:
a housing comprising an open first end, a second end, and a sidewall extending between the first end and the second end, and a protrusion comprising an open first end and a sidewall extending from the second end of the housing towards the open first end generally parallel to the sidewall of the housing configured to engage the male connector for retaining the male connector within the housing, and a center post extending from the center of the second end of the housing towards the center of the open first end of the housing configured to be inserted into and block the central lumen of male connector;
an outer absorbent support disposed in the housing between the sidewall of the housing and the sidewall of the protrusion configured to contain a cleaning solution for cleaning and disinfecting portions of the male connector engaged to the cap including the annular shield of the male connector; and
an inner absorbent support disposed between the protrusion and the center post configured to contain a cleaning solution for cleaning and disinfecting portions of the male connector engaged to the cap.

18. The cap of claim 17, wherein the housing including the protrusion and the center post comprises a rigid thermoplastic polymer, comprising at least one of polyester, polycarbonate, polypropylene, polyethylene, polyethylene terephthalate, or acrylonitrile butadiene styrene; and
Wherein the outer absorbent support and the inner absorbent support comprise sponges.

19. The cap of claim 17, wherein the male connector comprises:
a male luer connector comprising a luer stem comprising a tip and defining the central lumen and the shield extends about the stem; and
wherein the shield of the male luer connector comprises threads on an inner surface of the shield configured to engage corresponding threads of a female luer connector for securing the male connector to the female luer connector.

20. The cap of claim 18, wherein when the male connector is engaged to the cap:
the shield of the male connector is disposed in a recess defined by the sidewall of the housing and the sidewall of the protrusion such that the shield contacts and axially compresses the outer absorbent support thereby releasing cleaning solution from the outer absorbent support that wets and thereby cleans and disinfects the shield of the male connector;
the threads on the inner surface of the shield engage a thread extending from the sidewall of the protrusion thereby releasably connecting the male connector to the cap;
the luer stem of the male connector is disposed in a recess defined by the sidewall of the protrusion and the center post such that the luer stem contacts and axially compresses the inner absorbent support thereby releasing cleaning solution from the inner absorbent support that wets and thereby cleans and disinfects the outside of the luer stem of the male connector including the tip of the luer stem; and
the center post is disposed within the central lumen of male connector thereby blocking entry of the cleaning solution into the lumen.
